(19) United States
(12) Patent Application Publication
(10) Pub. No.: US 2024/0199669 A1
(43) Pub. Date: Jun. 20, 2024

(54) SELF-ASSEMBLED COMPLEX INCLUDING ONE OR MORE OF COBALT, SILVER, GADOLINIUM, OR IRON

(71) Applicant: Korea University Research and Business Foundation, Seoul (KR)

(72) Inventors: Heemin KANG, Seoul (KR); Sungkyu LEE, Siheung-si (KR); Seong Yeol KIM, Seoul (KR); Yuri KIM, Seoul (KR)

(73) Assignee: Korea University Research and Business Foundation, Seoul (KR)

(21) Appl. No.: 18/538,082

(22) Filed: Dec. 13, 2023

(30) Foreign Application Priority Data
Dec. 19, 2022 (KR) 10-2022-0178640

(51) Int. Cl. C07F 15/06; C07F 15/02

(52) U.S. Cl. CPC C07F 15/06; C07F 15/02

(57) ABSTRACT

The present disclosure relates to a self-assembled complex composed of substances existing in a body and using the same as ligands, the self-assembled complex including: metal ions including one or more of cobalt (Co), silver (Ag), gadolinium (Gd), or iron (Fe); and one or more ligands that are self-assembled with the metal ions, wherein: the self-assembled complex is reversibly self-assembled or self-disassembled; self-assembly is performed by one or more of a concentration of the metal ions or a concentration of the ligand; and the form of the self-assembled complex is controlled depending on a combination of the ligands.

### CROSS-REFERENCE TO RELATED APPLICATIONS

This application claims priority to and the benefit of Korean Patent Application No. 10-2022-0178640 filed in the Korean Intellectual Property Office on Dec. 19, 2022, the entire contents of which are incorporated herein by reference.

### TECHNICAL FIELD

The present disclosure relates to a self-assembled complex including one or more of cobalt, silver, gadolinium, or iron, and more specifically, to a self-assembled complex including one or more of cobalt, silver, gadolinium, or iron capable of controlling a shape and size by using substances existing in the body as ligands and capable of delivering the substances.

### BACKGROUND ART

Recently, the interest in anti-virus and anti-bacteria has increased due to the global COVID pandemic, and antiviral and antibacterial technologies using metal ions have been developed. Silver and cobalt affect the metabolism of cells or viruses, causing cell death and destruction of viruses.

The main products and technologies are those that enjoy antibacterial and antiviral effects through the release of metal ions by applying the metal ions to places that people frequently come into contact with.

Copper ions, which are mainly used for antibacterial and antiviral purposes, may cause toxicity to the human body such as hepatotoxicity, nephrotoxicity, anemia, immunotoxicity, and developmental toxicity, and thus may be fatal to infants, pregnant women, and the elderly. Accordingly, the copper ions need to be replaced with other metals.

Cobalt ions are a rare example of being used in a complex within the body, and form a complex with glyoxime ligand and have physiological functions as vitamin B12. This has a high possibility of being used in the body and has low toxicity in the body. However, the cobalt ions are difficult to apply to various substances and are not easily formed as a complex in the body.

In addition, there are products that use silver nanoparticles for antibacterial purposes, but the nanoparticle synthesis price competitiveness of silver ions is lacking.

Aging is progressing globally. As the human body ages, the probability of developing cancer increases and the social costs of cancer are increasing. Diagnosing and treating cancer at an early stage is becoming important. Research on theragnosis, which may perform both diagnosis and treatment at the same time, is active, and the need for materials that may both diagnose and treat cancer is emerging.

The substances used in theragnosis using MRI need to have low toxicity in the body and need to be able to target cancer and treat the same efficiently. It mainly uses an antigen-antibody reaction to specifically bind to cancer tissue, but this requires additional processing of theragnosis materials or requires additional substances such as peptides and proteins, making it less cost-competitive and difficult to be generalized to the public.

Accordingly, various studies are being conducted on materials that may be self-assembled with substances in the body, have high antibacterial properties, are not toxic to the human body, and have low production costs, and materials that may diagnose and treat various diseases as well as anti-cancer.

### DISCLOSURE

### Technical Problem

An aspect of the present disclosure is directed to providing a self-assembled complex capable of self-assembly using substances existing in the body.

In addition, another aspect of the present disclosure is directed to providing a self-assembled complex that is non-toxic to the human body and whose shape and size may be controlled in various ways.

In addition, yet another aspect of the present disclosure is directed to providing a self-assembled complex including one or more of cobalt, silver, gadolinium, or iron capable of self-assembly with substances in the body and having high antibacterial properties and low production costs.

### Solution to Problem

According to one aspect of the present invention, embodiments of the present invention may be a self-assembled complex composed of substances existing in a body and using the same as ligands, the self-assembled complex comprising: metal ions comprising one or more of cobalt (Co), silver (Ag), gadolinium (Gd), or iron (Fe); and one or more ligands that are self-assembled with the metal ions: the self-assembled complex is reversibly self-assembled or self-disassembled; self-assembly is performed by one or more of a concentration of the metal ions or a concentration of the ligand; and a form of the self-assembled complex is controlled depending on a combination of the ligands.

In one embodiment, the metal ion may be a cobalt ion, a silver ion, a gadolinium ion, or a mixture of gadolinium ion and iron ion.

In one embodiment, the self-assembled complex may be self-disassembled by an external solution containing external ions.

In one embodiment, the self-assembled complex may be provided as a structure by aggregating one or more of the metal ions and the ligand.

In one embodiment, the ligand may comprise one or more of phosphate or phosphonate.

In one embodiment, the ligand may comprise one or more of adenosine monophosphate, adenosine diphosphate, or adenosine triphosphate.

In one embodiment, the ligand may comprise one or more phosphates; the self-assembled complex may be provided as a structure by aggregating one or more of the metal ions and the ligand; the structure may be provided in a helical shape; and a size and the number of helices of the structure may be controlled depending on a degree of mixing of the ligand with respect to the number of the phosphates.

In one embodiment, the ligand may comprise one or more phosphates; the self-assembled complex may be provided as a structure by aggregating one or more of the metal ions and the ligand; the structure may be provided in one or more of a helical, rod, or spherical shape; and a form of the structure may be controlled depending on a degree of mixing of the ligand with respect to the number of the phosphates.

In one embodiment, the ligand may comprise one or more phosphates; the self-assembled complex may be provided as a structure by aggregating one or more of the metal ions and the ligand; the structure may be provided in a rod shape with an aspect ratio; and the aspect ratio of the structure may be controlled depending on a degree of mixing of the ligand with respect to the number of the phosphates.

In one embodiment, the ligand may comprise one or more phosphates; the self-assembled complex may be provided as a structure by aggregating one or more of the metal ions and the ligand; and a degree of aggregation or a size of the structure may be controlled depending on a degree of mixing of the ligand with respect to the number of the phosphates.

In one embodiment, the self-assembled complex further comprising an active ingredient therein, the active ingredient comprises one or more of adenosine, guanosine, uridine, cytidine, or doxorubicin.

In one embodiment, the metal ions may include ions of any one of cobalt (Co), gadolinium (Gd), or iron (Fe); and the self-assembled complex has paramagnetic properties.

In one embodiment, the metal ions may include ions of any one of cobalt (Co), gadolinium (Gd), or iron (Fe); and the self-assembled complex moves as much as a first distance when an externally applied magnetic field is applied.

In one embodiment, the metal ions may include ions of any one of cobalt (Co), gadolinium (Gd), or iron (Fe); and the self-assembled complex has paramagnetic properties, and a magnetic moment is adjusted depending on a condition formed.

In one embodiment, the self-assembled complex may be formed between the metal ions and the ligand through a coordination bond; and neighboring ligands are connected through π-π interaction or hydrogen bonding.

In one embodiment, the metal ion may be a gadolinium (Gd) ion or a mixture of gadolinium (Gd) and iron (Fe) ions; the self-assembled complex is provided as a structure by aggregating the metal ions and the ligand; and a size of the structure is controlled depending on a concentration of the ligand.

In one embodiment, the metal ion may be a gadolinium (Gd) ion or a mixture of gadolinium (Gd) and iron (Fe) ions; the self-assembled complex is provided as a structure by aggregating the metal ions and the ligand; and a size of the structure is controlled depending on a reaction time.

In one embodiment, the metal ion may be a gadolinium (Gd) ion or a mixture of gadolinium (Gd) and iron (Fe) ions; and the self-assembled complex is loaded with a drug of doxorubicin or adenosine, and the drug continuously exhibits a release pattern for a desired period of time, resulting in sustained-release release.

In one embodiment, the desired period of time may be a period selected from 0.5 hours to 12 months.

In one embodiment, a release rate of the drug may be controlled depending on a size of the self-assembled complex.

### Advantageous Effects

According to an embodiment of the present disclosure as described above, it is possible to provide a self-assembled complex capable of self-assembly using materials existing in the body.

In addition, according to an embodiment of the present disclosure, it is possible to provide a self-assembled complex that is not toxic to the human body and whose shape and size can be controlled in various ways.

### BRIEF DESCRIPTION OF DRAWINGS

FIG. 1 is a diagram schematically illustrating a self-assembled complex using cobalt metal ions and adenosine monophosphate or mixed adenosine phosphate ligands among the phosphate ligands in an embodiment of the present disclosure.

FIG. 2 is a diagram illustrating cobalt metal ions and adenosine monophosphate self-assembled to form a helical self-assembled complex.

FIG. 3 is a diagram illustrating the aspect ratio control of a self-assembled complex with a mixed ligand of cobalt metal ions, adenosine monophosphate, and adenosine triphosphate.

FIG. 4 is a diagram identifying the element ratio of the self-assembled complex of cobalt metal ions and adenosine monophosphate using a transmission electron microscope.

FIG. 5 is a diagram illustrating the movement of a self-assembled complex using cobalt metal ions and adenosine monophosphate in an embodiment of the present disclosure when an external magnetic field is applied.

FIG. 6 is a diagram schematically illustrating a self-assembled complex using silver metal ions and adenosine monophosphate or mixed adenosine phosphate ligands among the phosphate ligands in an embodiment of the present disclosure.

FIG. 7 is a diagram schematically illustrating the formation of a self-assembled complex of gadolinium ions and a ligand, and the formation of a self-assembled complex of gadolinium, iron ions, and a ligand, and mass transfer using the same according to an embodiment of the present disclosure.

FIG. 8 is a diagram illustrating a self-assembled complex depending on a concentration of gadolinium ions and a ligand according to an embodiment of the present disclosure.

FIG. 9 is a diagram illustrating the effect of quenching on a self-assembled complex of gadolinium and iron ions and a ligand.

FIG. 10 is a diagram illustrating the movement of a self-assembled complex of gadolinium ions and a ligand, and a self-assembled complex of gadolinium, iron ions, and a ligand when an external magnetic field is applied.

### DESCRIPTION OF EMBODIMENTS

The specific details of other embodiments are included in the detailed description and drawings.

Advantages and features of the present disclosure, and a method to achieve the same will become apparent with reference to the embodiments described below in detail in conjunction with the accompanying drawings. However, the present disclosure is not limited to the embodiments disclosed below and may be implemented in a variety of different forms. Unless otherwise specified in following descriptions, all of numbers, values, and/or expressions indicating ingredients, reaction conditions, and contents of ingredients in the present disclosure are, in essence, approximations thereof based on various uncertainties in measurements which occur in obtaining the numbers, values, and/or expressions. Thus, the numbers, values and/or expressions should be understood as being modified by a term “about” in all instances. Further, where a numerical range is disclosed in the present description, the range is continuous and includes a minimum value and a maximum value of the range, unless otherwise indicated. Further, where the number or the value refers to an integer, the range includes all of integers included between the minimum and the maximum of the range, unless otherwise indicated.

Further, in the present disclosure, when a variable is included in a range, the variable will be understood to include all values within a stated range including stated endpoints of the range. For example, a range of “5 to 10” includes values of 5, 6, 7, 8, 9, and 10, as well as any subranges such as 6 to 10, 7 to 10, 6 to 9, 7 to 9, etc. It will be understood that the variable includes any value between valid integers in a stated range such as 5.5, 6.5, 7.5, 5.5 to 8.5, and 6.5 to 9, etc. For example, a range “10% to 30%” includes all of integer values such as 10%, 11%, 12%, 13%, 30%, etc. as well as any subranges such as 10% to 15%, 12% to 18%, or 20% to 30%, etc. It will be understood that the range includes any value between valid integers within the stated range such as 10.5%, 15.5%, 25.5%, etc.

An embodiment of the present disclosure provides a self-assembled complex composed of substances existing in a body and using the same as ligands, the self-assembled complex including: metal ions including one or more of cobalt (Co), silver (Ag), gadolinium (Gd), or iron (Fe); and one or more ligands that are self-assembled with the metal ions, wherein the self-assembled complex is reversibly self-assembled or self-disassembled, self-assembly is performed by one or more of a concentration of the metal ions or a concentration of the ligand, and the form of the self-assembled complex is controlled depending on a combination of the ligands.

Specifically, the self-assembled complex may form a self-assembled complex with a metal ion at room temperature by using a substance existing in the body as a ligand. In addition, the self-assembled complex has low toxicity in the body and does not require additional chemical treatment, thereby providing cost-competitive antibacterial and antiviral properties.

The metal ion may be a cobalt ion, a silver ion, a gadolinium ion, or a mixture of gadolinium ion and iron ion.

When the metal ion is cobalt, the self-assembled complex may be formed into a helical structure when having paramagnetic properties. When the metal ion is silver, the self-assembled complex may be formed into a spherical structure, and the size of the sphere may be controlled. When the metal ion is gadolinium, the self-assembled complex may have paramagnetic properties, may be controlled in size, and may be used as an MRI contrast agent. When the metal ion is a mixture of gadolinium and iron, the self-assembled complex may have paramagnetic properties, may be controlled in size, and may be used as an MRI contrast agent.

Specifically, in the self-assembled complex, when the metal ion is either a gadolinium (Gd) ion or a mixture of gadolinium (Gd) ions and iron (Fe) ions, the self-assembled complex may have magnetic properties and may be used as an MRI contrast agent. Specifically, the gadolinium (Gd) ion or a mixture of gadolinium (Gd) ions and iron (Fe) ions is self-assembled using a substance existing in the body as a ligand to form a self-assembled complex. The self-assembled complex not only makes a magnetic material with low toxicity in the body and capable of diagnosis, but also allows drugs to be easily loaded into the material. Accordingly, the self-assembled complex may be used as a material for diagnosing and treating various diseases as well as anti-cancer.

The self-assembled complex may be self-disassembled by an external solution containing external ions. Specifically, the external solution may include a buffer solution. The self-assembled complex according to this embodiment is spontaneously self-assembled and self-disassembled, but self-disassembly may be promoted under conditions that further include the buffer solution. The buffer solution may create an environment similar to that in a living body, and the buffer solution may promote disassembly of the self-assembled complex by exchanging ions with the components of the self-assembled complex.

The self-assembled complex may be provided as a structure by aggregating one or more of the metal ions and the ligand.

The ligand may include one or more of phosphate or phosphonate, and in particular, the ligand may include one or more of adenosine monophosphate, adenosine diphosphate, or adenosine triphosphate.

For example, the ligand may be at least one of AMP (Adenosine monophosphate), ADP (Adenosine diphosphate), ATP (Adenosine triphosphate), TMP (Thymidine monophosphate), TDP (Thymidine diphosphate), TTP (Thymidine triphosphate), CMP (Cytidine monophosphate), CDP (Cytidine diphosphate), CTP (Cytidine triphosphate), GMP (Guanosine monophosphate), GDP (Guanosine diphosphate), GTP (Guanosine triphosphate), UMP (Uridine monophosphate), UDP (Uridine diphosphate), UTP (Uridine triphosphate), DNA, RNA, AEP (2-aminoethylphosphonic acid), TNA (Threose nucleic acid), GNA (glycol nucleic acid), HNA (1,5-anhydrohexitol nucleic acid), ANA (1,5-anhydroatritol nucleic acid), FANA (2′-deoxy-2′-fluoroarabino nucleic acid), or CeNA (cyclohexenyl nucleic acid).

The ligand may include one or more phosphates, and the self-assembled complex may be provided as a structure by aggregating one or more of the metal ions and the ligand.

When the metal ion is a cobalt ion, the structure may be provided in a helical shape, and a size and the number of helices of the structure may be controlled depending on a degree of mixing of the ligand with respect to the number of the phosphates.

When the metal ion is one or more of cobalt, silver, gadolinium, and iron, the structure may be provided in one or more of a helical, rod, or spherical shape. In this connection, the form of the structure may be controlled depending on the degree of mixing of the ligand with respect to the number of phosphates.

When the structure is provided in a rod shape with an aspect ratio, the aspect ratio of the structure may be controlled depending on the degree of mixing of the ligand with respect to the number of phosphates.

For example, in a structure formed by aggregation of the metal ion and the ligand, the degree of aggregation or size of the structure may be controlled depending on the degree of mixing of the ligand with respect to the number of phosphates included in the ligand.

The self-assembled complex according to this embodiment may further include an active ingredient therein, and the active ingredient may be one or more of adenosine, guanosine, uridine, cytidine, or doxorubicin.

The active ingredient may be supported on the self-assembled complex. The active ingredient may be assembled together when the self-assembled complex is self-assembled and supported within the self-assembled complex.

The active ingredient may be released when the self-assembled complex is self-disassembled. Accordingly, the release time and speed of the active ingredient may be adjusted by adjusting the time at which the self-assembled complex is self-assembled. The self-assembled complex may have a different speed of self-disassembly depending on the mixing ratio of the ligand, and the self-assembled complex may be sequentially self-disassembled at a constant speed from the surface. Accordingly, the active ingredient may be released consistently and continuously during the time when the self-assembled complex is self-disassembled. In other words, the active ingredient may be released consistently and continuously during the release time.

When the metal ions include ions of any one of cobalt (Co), gadolinium (Gd), or iron (Fe), the self-assembled complex may have paramagnetic properties.

When the metal ions include ions of any one of cobalt (Co), gadolinium (Gd), or iron (Fe), the self-assembled complex may move as much as a first distance when an externally applied magnetic field is applied.

When the metal ion is any one of cobalt (Co), gadolinium (Gd), or iron (Fe), the self-assembled complex may have paramagnetic properties, and a magnetic moment may be adjusted depending on a condition formed.

The self-assembled complex may be formed between the metal ions and the ligand through a coordination bond, and neighboring ligands may be connected through T-T interaction or hydrogen bonding.

For example, the metal ion may form a metal complex by coordinating bond with a phosphate group of the ligand, AMP or ATP. Since a base portion of the ligand includes an aromatic ring, a π-π interaction may be formed between aromatic rings. A water molecule may bind to the metal ion, and the water molecule may form a hydrogen bond with nitrogen of the base portion.

The metal ion may be a gadolinium (Gd) ion or a mixture of gadolinium (Gd) and iron (Fe) ions, the self-assembled complex may be provided as a structure by aggregating the metal ion and the ligand, and a size of the structure may be controlled depending on a concentration of the ligand. In addition, the size of the structure may be controlled depending on a reaction time.

In an embodiment of the present disclosure, when the metal ion is gadolinium (Gd) ion or a mixture of gadolinium (Gd) and iron (Fe) ions, the self-assembled complex may be loaded with a drug of doxorubicin or adenosine, and the drug may continuously exhibit a release pattern for a desired period of time, resulting in sustained-release release. The desired period of time may be a period selected from 0.5 hours to 12 months, and a release rate of the drug may be controlled depending on a size of the self-assembled complex.

Hereinafter, examples and comparative examples of the present disclosure will be described. However, the following examples are only preferred embodiments of the present disclosure and the scope of right of the present disclosure is not limited by the following examples.

### [Preparation Examples]

### 1. Preparation of Self-Assembled Complex Including Cobalt Ions

Cobalt chloride, AMP (Adenosine monophosphate), and ATP (Adenosine triphosphate) are each dissolved in ultrapure water so that the final concentration becomes 1 M. A stock solution is put in a conical tube, and the amount of ultrapure water is calculated for addition. Herein, adenosine phosphate aqueous solution and cobalt chloride aqueous solution are added in that order, mixed well by vortexing, and reacted for 24 hours. When the reaction for 24 hours is completed, an unreacted material is removed, and the self-assembly is obtained by centrifugation at 10,000 RPM for 10 minutes using a centrifuge to settle a self-assembly and remove the supernatant. The ultrapure water is added in an equal volume to the original volume and centrifuged again. This process is repeated twice.

### 2. Preparation of Self-Assembled Complex Including Silver Ions

Silver nitrate, AMP (Adenosine monophosphate), and ATP (Adenosine triphosphate) are each dissolved in ultrapure water so that the final concentration becomes 1 M. A stock solution is put in a conical tube, and the amount of ultrapure water is calculated for addition. Herein, adenosine phosphate aqueous solution and silver nitrate aqueous solution are added in that order, mixed well by vortexing, and reacted for 24 hours. When the reaction for 24 hours is completed, an unreacted material is removed, and the self-assembly is obtained by centrifugation at 10,000 RPM for 10 minutes using a centrifuge to settle a self-assembly and remove the supernatant. The ultrapure water is added in an equal volume to the original volume and centrifuged again. This process is repeated twice.

### 3. Preparation of Self-Assembled Complex Including Gadolinium Ions

Gadolinium chloride, AMP (Adenosine monophosphate), and ATP (Adenosine triphosphate) are each dissolved in ultrapure water so that the final concentration becomes 1 M. A stock solution is put in a conical tube, and the amount of ultrapure water is calculated for addition. Herein, adenosine phosphate aqueous solution and gadolinium chloride aqueous solution are added in that order, mixed well by vortexing, and reacted for 24 hours. When the reaction for 24 hours is completed, an unreacted material is removed, and the self-assembly is obtained by centrifugation at 10,000 RPM for 10 minutes using a centrifuge to settle a self-assembly and remove the supernatant. The ultrapure water is added in an equal volume to the original volume and centrifuged again. This process is repeated twice.

### 4. Preparation of Self-Assembled Complex Including Gadolinium-Iron Ions

Gadolinium chloride, ferric chloride, AMP (Adenosine monophosphate), and ATP (Adenosine triphosphate) are each dissolved in ultrapure water so that the final concentration becomes 1 M. A stock solution is put in a conical tube, and the amount of ultrapure water is calculated for addition. Herein, adenosine phosphate aqueous solution, gadolinium chloride aqueous solution, and ferric chloride aqueous solution are added in that order, mixed well by vortexing, and reacted for 24 hours or 10 minutes. When the reaction is completed, an unreacted material is removed, and the self-assembly is obtained by centrifugation at 10,000 RPM for 10 minutes using a centrifuge to settle a self-assembly and remove the supernatant. The ultrapure water is added in an equal volume to the original volume and centrifuged again. This process is repeated twice.

5. Substances used in Experiments
Cobalt(II) chloride, (CAS: 7646-79-9, Sigma-Aldrich)
Silver(I) nitrate, (CAS: 7761-88-8, DAEJUNG Chemicals & Metals)
Gadolinium(III) chloride hexahydrate (CAS: 13450-84-5, Sigma-Aldrich)
Iron(III) chloride hexahydrate (CAS: 10025-77-1, Sigma-Aldrich)
Adenosine-5′-monophosphate disodium salt (CAS: 4578-31-8, Alfa Aesar)
Adenosine 5′-triphosphate disodium salt (CAS: 51963-61-2, DAEJUNG Chemicals & Metals)

### EXAMPLES

### Example 1

A mixed solution was prepared with cobalt chloride while changing the concentrations of AMP and ATP in 10 ml. Thereafter, the reaction was performed at room temperature for 24 hours to form a self-assembled complex.

TABLE 1

| | AMP | | ATP | | Cobalt chloride |
|---|---|---|---|---|---|
| | concentration | | concentration | | concentration |
| Example 1-1 | 25 | mM | 0 | mM | 25 mM |
| Example 1-2 | 24.75 | mM | 0.25 | mM | 25 mM |

### Example 2

A mixed solution was prepared with cobalt chloride while changing the concentrations of AMP and ATP in 10 ml. Thereafter, the reaction was performed at room temperature for 24 hours to form a self-assembled complex.

TABLE 2

| | AMP | | ATP | | Cobalt chloride |
|---|---|---|---|---|---|
| | concentration | | concentration | | concentration |
| Example 2-1 | 22.5 | mM | 2.5 | mM | 25 mM |
| Example 2-2 | 21.25 | mM | 3.75 | mM | 25 mM |
| Example 2-2 | 20 | mM | 5 | mM | 25 mM |

### Example 3

A mixed solution was prepared with silver nitrate while changing the concentrations of AMP and ATP in 10 ml. Thereafter, the reaction was performed at room temperature for 24 hours to form a self-assembled complex.

TABLE 3

| | AMP | | ATP | | Silver nitrate |
|---|---|---|---|---|---|
| | concentration | | concentration | | concentration |
| Example 3-1 | 50 | mM | 0 | mM | 50 mM |
| Example 3-2 | 45 | mM | 5 | mM | 50 mM |
| Example 3-3 | 25 | mM | 25 | mM | 50 mM |
| Example 3-4 | 5 | mM | 45 | mM | 50 mM |
| Example 3-4 | 0 | mM | 50 | mM | 50 mM |

### Example 4

A mixed solution was prepared while changing the concentrations of gadolinium chloride, AMP and ATP in 10 ml. Thereafter, the reaction was performed at room temperature for 24 hours to form a self-assembled complex.

TABLE 4

| | | | | | Gadolinium | |
|---|---|---|---|---|---|---|
| | AMP | | ATP | | chloride | |
| | concentration | | concentration | | concentration | |
| Example 4-1 | 0.5 | mM | 0.5 | mM | 1 | mM |
| Example 4-2 | 0.25 | mM | 0.25 | mM | 0.5 | mM |
| Example 4-3 | 0.05 | mM | 0.05 | mM | 0.1 | mM |

### Example 5

A mixed solution was prepared while maintaining the concentrations of gadolinium chloride, ferric chloride, AMP and ATP in 10 ml. Thereafter, the reaction time was adjusted at room temperature to form a self-assembled complex.

TABLE 5

| | | | Gadolinium | Ferric | |
|---|---|---|---|---|---|
| | AMP | ATP | chloride | chloride | Reaction |
| | concentration | concentration | concentration | concentration | time |
| Example 4-1 | 0.05 mM | 0.05 mM | 0.05 mM | 0.05 mM | 24 hours |
| Example 4-2 | 0.05 mM | 0.05 mM | 0.05 mM | 0.05 mM | 10 minutes |

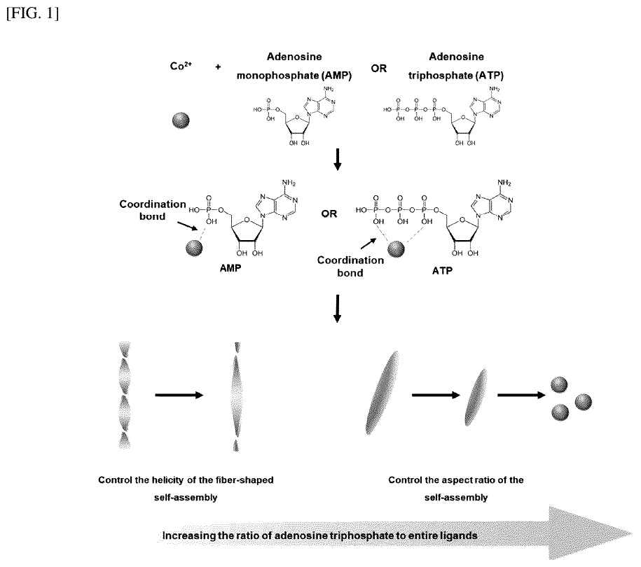

FIG. 1 is a diagram schematically illustrating a self-assembled complex using cobalt metal ions and adenosine monophosphate or mixed adenosine phosphate ligands among the phosphate ligands in an embodiment of the present disclosure. Cobalt metal ions and adenosine monophosphate are self-assembled to form a helical self-assembled complex, and as adenosine triphosphate is included in the self-assembled complex, the helicity decreases. As the concentration of adenosine triphosphate increases, a rod-shaped self-assembled complex is formed, and as the concentration of adenosine triphosphate increases, a spherical self-assembled complex is formed.

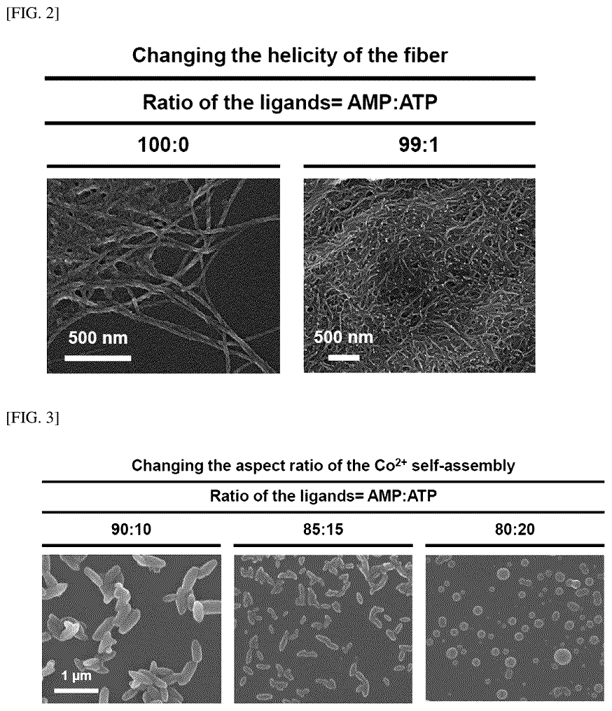

FIG. 2 shows that cobalt metal ions and adenosine monophosphate are self-assembled to form a helical self-assembled complex, and as adenosine triphosphate is included in the self-assembled complex, the helicity decreases. The form of the self-assembled complex may be identified through a scanning electron microscope.

FIG. 3 shows that the aspect ratio is controlled when a self-assembled complex is formed with a mixed ligand of cobalt metal ions, adenosine monophosphate, and adenosine triphosphate. As the concentration of adenosine triphosphate increases, the aspect ratio converges to 1, resulting in a spherical self-assembled complex. The form of the self-assembled complex may be identified through a scanning electron microscope.

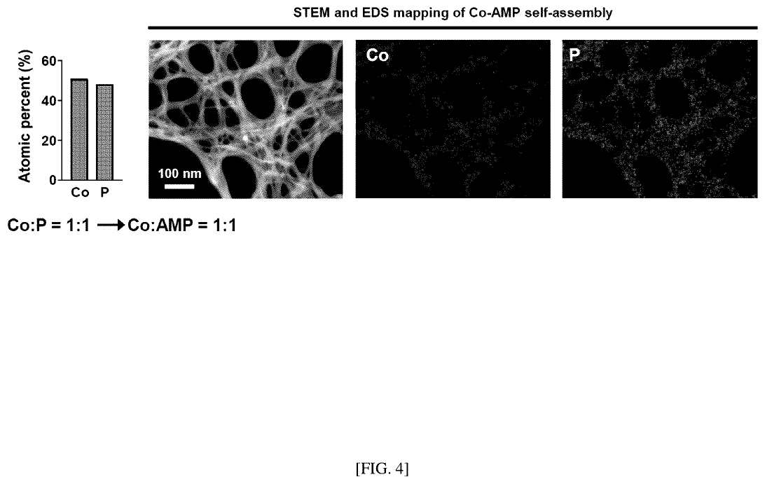

FIG. 4 identifies the element ratio of the self-assembled complex of cobalt metal ions and adenosine monophosphate using a transmission electron microscope. Cobalt metal ions and adenosine monophosphate are present in a 1:1 ratio in the self-assembled complex.

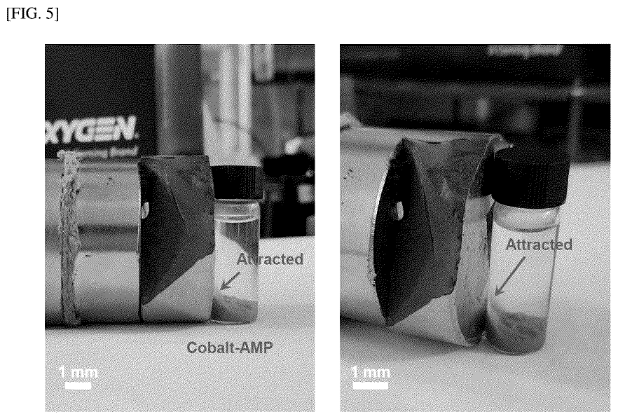

FIG. 5 shows that when an external magnetic field is applied to a self-assembled complex using cobalt metal ions and adenosine monophosphate in an embodiment of the present disclosure, it may be identified that the self-assembled complex is attracted in the direction of the magnet within 2 hours after the external magnetic field is applied while the self-assembled complex is dispersed in water. The paramagnetic property of the cobalt self-assembled complex was measured using a vibrating sample magnetometer.

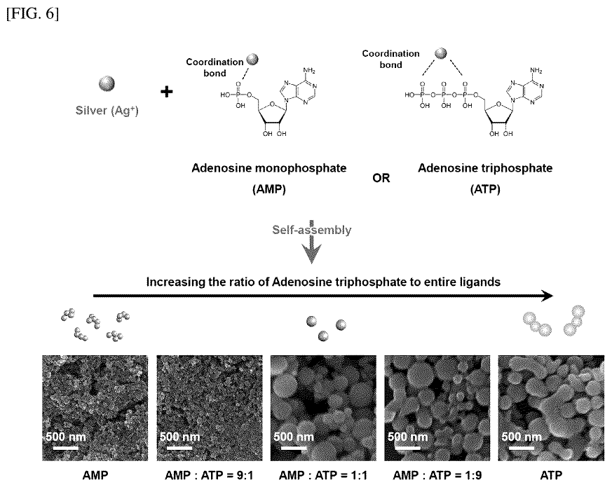

FIG. 6 is a diagram schematically illustrating a self-assembled complex using silver metal ions and adenosine monophosphate or mixed adenosine phosphate ligands among the phosphate ligands in an embodiment of the present disclosure. The form of the self-assembled complex may be identified through a scanning electron microscope. It was identified that the size of a single self-assembled complex increases as adenosine triphosphate is included in the self-assembled complex.

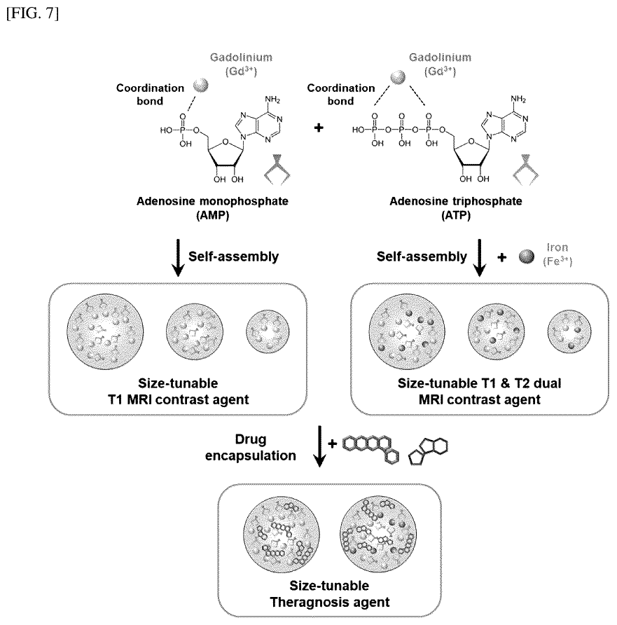

FIG. 7 a diagram schematically illustrating: a magnetic self-assembled complex capable of being used as a T1 contrast agent using gadolinium metal ions and adenosine monophosphate or mixed adenosine phosphate ligand among the phosphate ligands; a magnetic self-assembled complex capable of being used as a T1 and T2 contrast agent by mixing gadolinium metal ions and iron metal ions and using adenosine monophosphate or mixed adenosine phosphate ligands among the phosphate ligands; and a self-assembled complex that is loaded with various drugs at the time of forming the self-assembled complex, enables local targeting using magnetism, and allows diagnosis and treatment at the same time in an embodiment of the present disclosure.

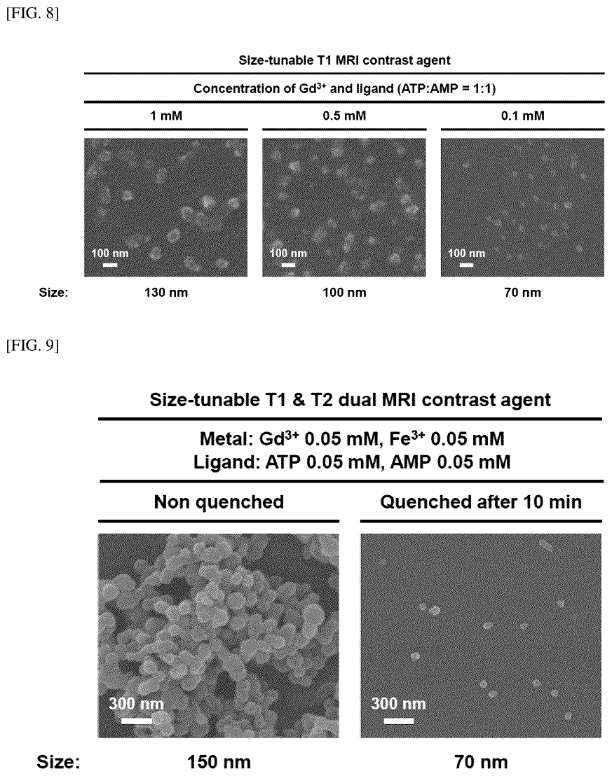

FIG. 8 shows an embodiment of the present disclosure in which the size of the self-assembled complex is controlled by simultaneously adjusting the concentration of gadolinium metal ion and ligand, and this may be identified through a scanning electron microscope.

FIG. 9 shows an embodiment of the present disclosure in which gadolinium and iron metal ions are mixed and reacted with a ligand to form a self-assembled complex. The size of the self-assembled complex may be controlled by quenching during the reaction, and this may be identified through a scanning electron microscope.

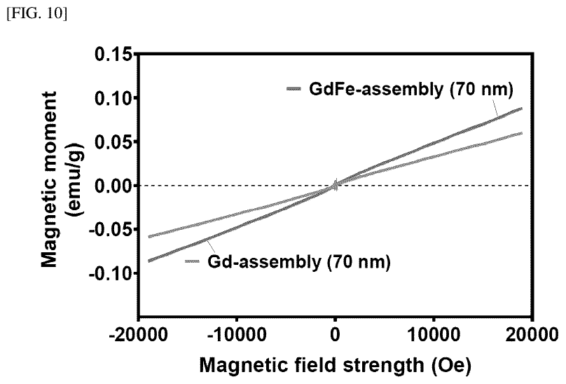

FIG. 10 shows an embodiment of the present disclosure in which the paramagnetic properties of the self-assembled complex using gadolinium and the self-assembled complex using gadolinium and iron are measured using a vibrating sample magnetometer. In addition, it was identified that the self-assembled complex using gadolinium was capable of loading drugs, and that the loaded drug was released from the buffer solution for several days.

It will be appreciated that the technical configuration of the present disclosure as described above may be practiced by those skilled in the art to which the present disclosure pertains in other specific forms without departing from the technical spirit or essential characteristics of the present disclosure. It is, therefore, to be understood that the embodiments as described above are to be considered in all respects only as illustrative and not restrictive. The scope of the present disclosure is defined by the appended claims rather than the above detailed description. All changes or modifications that come within the meaning and range of equivalency of the claims, and equivalents thereof, are to be construed as being included within the scope of the present disclosure.

1. A self-assembled complex composed of substances existing in a body and using the same as ligands, the self-assembled complex comprising:
metal ions comprising one or more of cobalt (Co), silver (Ag), gadolinium (Gd), or iron (Fe); and
one or more ligands that are self-assembled with the metal ions, wherein:
the self-assembled complex is reversibly self-assembled or self-disassembled;
self-assembly is performed by one or more of a concentration of the metal ions or a concentration of the ligand; and
a form of the self-assembled complex is controlled depending on a combination of the ligands.

2. The self-assembled complex of claim 1, wherein the metal ion is a cobalt ion, a silver ion, a gadolinium ion, or a mixture of gadolinium ion and iron ion.

3. The self-assembled complex of claim 1, wherein the self-assembled complex is self-disassembled by an external solution containing external ions.

4. The self-assembled complex of claim 1, wherein the self-assembled complex is provided as a structure by aggregating one or more of the metal ions and the ligand.

5. The self-assembled complex of claim 1, wherein the ligand comprises one or more of phosphate or phosphonate.

6. The self-assembled complex of claim 5, wherein the ligand comprises one or more of adenosine monophosphate, adenosine diphosphate, or adenosine triphosphate.

7. The self-assembled complex of claim 1, wherein:
the ligand comprises one or more phosphates;
the self-assembled complex is provided as a structure by aggregating one or more of the metal ions and the ligand;
the structure is provided in a helical shape; and
a size and the number of helices of the structure is controlled depending on a degree of mixing of the ligand with respect to the number of the phosphates.

8. The self-assembled complex of claim 1, wherein:
the ligand comprises one or more phosphates;
the self-assembled complex is provided as a structure by aggregating one or more of the metal ions and the ligand;
the structure is provided in one or more of a helical, rod, or spherical shape; and
a form of the structure is controlled depending on a degree of mixing of the ligand with respect to the number of the phosphates.

9. The self-assembled complex of claim 1, wherein:
the ligand comprises one or more phosphates;
the self-assembled complex is provided as a structure by aggregating one or more of the metal ions and the ligand;
the structure is provided in a rod shape with an aspect ratio; and
the aspect ratio of the structure is controlled depending on a degree of mixing of the ligand with respect to the number of the phosphates.

10. The self-assembled complex of claim 1, wherein:
the ligand comprises one or more phosphates;
the self-assembled complex is provided as a structure by aggregating one or more of the metal ions and the ligand; and
a degree of aggregation or a size of the structure is controlled depending on a degree of mixing of the ligand with respect to the number of the phosphates.

11. The self-assembled complex of claim 1, further comprising an active ingredient therein, wherein the active ingredient comprises one or more of adenosine, guanosine, uridine, cytidine, or doxorubicin.

12. The self-assembled complex of claim 1, wherein:
the metal ions include ions of any one of cobalt (Co), gadolinium (Gd), or iron (Fe); and
the self-assembled complex has paramagnetic properties.

13. The self-assembled complex of claim 1, wherein:
the metal ions include ions of any one of cobalt (Co), gadolinium (Gd), or iron (Fe); and
the self-assembled complex moves as much as a first distance when an externally applied magnetic field is applied.

14. The self-assembled complex of claim 1, wherein:
the metal ions include ions of any one of cobalt (Co), gadolinium (Gd), or iron (Fe); and
the self-assembled complex has paramagnetic properties, and a magnetic moment is adjusted depending on a condition formed.

15. The self-assembled complex of claim 1, wherein:
the self-assembled complex is formed between the metal ions and the ligand through a coordination bond; and
neighboring ligands are connected through π-π interaction or hydrogen bonding.

16. The self-assembled complex of claim 1, wherein:
the metal ion is a gadolinium (Gd) ion or a mixture of gadolinium (Gd) and iron (Fe) ions;
the self-assembled complex is provided as a structure by aggregating the metal ions and the ligand; and
a size of the structure is controlled depending on a concentration of the ligand.

17. The self-assembled complex of claim 1, wherein:
the metal ion is a gadolinium (Gd) ion or a mixture of gadolinium (Gd) and iron (Fe) ions;
the self-assembled complex is provided as a structure by aggregating the metal ions and the ligand; and
a size of the structure is controlled depending on a reaction time.

18. The self-assembled complex of claim 1, wherein:
the metal ion is a gadolinium (Gd) ion or a mixture of gadolinium (Gd) and iron (Fe) ions; and
the self-assembled complex is loaded with a drug of doxorubicin or adenosine, and the drug continuously exhibits a release pattern for a desired period of time, resulting in sustained-release release.

19. The self-assembled complex of claim 18, wherein the desired period of time is a period selected from 0.5 hours to 12 months.

20. The self-assembled complex of claim 18, wherein a release rate of the drug is controlled depending on a size of the self-assembled complex.
